SEQUENCE LISTING (4 sequences)

SEQ ID NO: 1 - DNA, 912 bp, synthetic construct
  source 1..912: mol_type = other DNA
atgactttaaacttgtctaataaacggaatttaatccctttattggtggggacttcgtgtcttttcattagtaatgcttttgcagctgatgttgttccccatacggcaaaaccaggggaggtttggattcttcaaaatgaccgttcagatgaattcgagtttgaagatactcgtaaatgggacttccaaccaaacccatttcaaatttggacctgggaaaaagacaatgctatcgttgaggatggaaagttaaaattaaccgcttcttacgagaaacacaaccgtaagttcgaagatggctgtaatgagaaaacaatttatgatttcccattgttcttcaagtcaggaatggtcaaatccaaagcatcaggcgtctatggctactacgaagcgaaaatcaaaggtactgacctacacccaggcgtatcacctgccttctggatttacagcaatatcgatacagcgctgaagaaagaaggtgacatccaatatgctgagatcgacgtcgttgaacttcttcaggacgagactgatcctacccacagtgaccacaacctacacaacatcattgttaaagatggtaaaccacaatgggtacgccccaagccaaacccagaaacgcaccaaaaccttcacaacctaggctttgacccgcgtaaagactttcacatctatgcagttgatgtaacaccagaagacatcacctggtacgtagacggtcaagaggtgggcaagaagcagaacctatattggcacaaagagatgaacgttgctttatcactgggtatgcgtggcgagaagttcacccgatgggattgtaaccaattctatccagttgacgttgctgaagcagatagcggtctaccaacaactatggaagtagagtacgttcgtgtatggagcagagagtaa

SEQ ID NO: 2 - protein, 303 aa, synthetic construct
MTLNLSNKRNLIPLLVGTSCLFISNAFAADVVPHTAKPGEVWILQNDRSDEFEFEDTRKWDFQPNPFQIWTWEKDNAIVEDGKLKLTASYEKHNRKFEDGCNEKTIYDFPLFFKSGMVKSKASGVYGYYEAKIKGTDLHPGVSPAFWIYSNIDTALKKEGDIQYAEIDVVELLQDETDPTHSDHNLHNIIVKDGKPQWVRPKPNPETHQNLHNLGFDPRKDFHIYAVDVTPEDITWYVDGQEVGKKQNLYWHKEMNVALSLGMRGEKFTRWDCNQFYPVDVAEADSGLPTTMEVEYVRVWSRE

SEQ ID NO: 3 - DNA, 33 bp, synthetic construct
  source 1..33: mol_type = other DNA
ggaattccatatgactttaaacttgtctaataa

SEQ ID NO: 4 - DNA, 34 bp, synthetic construct
  source 1..34: mol_type = other DNA
ccgctcgagctctctgctccatacacgaacgtac

(19) United States
(12) Patent Application Publication
(10) Pub. No.: US 2026/0226441 A1
(43) Pub. Date: Aug. 6, 2026

(54) KAPPA-CARRAGEENASE CGK-GDSX478 FOR DIRECTIONALLY PREPARING KAPPA-CARRAGEENAN DISACCHARIDE

(71) Applicants: Shenzhen Institute of Guangdong Ocean University, SHENZHEN (CN); GUANGDONG OCEAN UNIVERSITY, Zhanjiang (CN)

(72) Inventors: Saiyi ZHONG, SHENZHEN (CN); Jing CHEN, SHENZHEN (CN); Huanming LIU, SHENZHEN (CN); Zhifei ZENG, SHENZHEN (CN); Liang JIANG, SHENZHEN (CN); Huiping LIU, SHENZHEN (CN); Xuewu ZHANG, SHENZHEN (CN); Zhuo WANG, SHENZHEN (CN); Jieliang ZHANG, SHENZHEN (CN); Mingxue PAN, SHENZHEN (CN)

(73) Assignees: Shenzhen Institute of Guangdong Ocean University, SHENZHEN (CN); GUANGDONG OCEAN UNIVERSITY, Zhanjiang (CN)

(21) Appl. No.: 19/411,204

(22) Filed: Dec. 6, 2025

Related U.S. Application Data
(63) Continuation of application No. PCT/CN2024/138549, filed on Dec. 11, 2024.

(30) Foreign Application Priority Data
Sep. 12, 2024 (CN) 202411273107.3
Dec. 6, 2024 (CN) 202411788452.0

(51) Int. Cl. C12N 9/24; C12N 15/70; C12P 19/12; C12P 19/14

(52) U.S. Cl. CPC C12N 9/2402; C12N 15/70; C12P 19/12; C12P 19/14; C12Y 302/01083

(57) ABSTRACT

The present disclosure discloses a kappa-carrageenase Cgk-GDSX478 for directionally preparing a kappa-carrageenan disaccharide and relates to the technical field of biological enzymes. A nucleotide sequence of a gene Cgk-GDSX478 encoding kappa carrageenase is shown in SEQ ID NO: 1, and the amino acid sequence of the encoding kappa carrageenase Cgk-GDSX478 is shown in SEQ ID NO: 2. The present disclosure also provides a method for preparing the kappa-carrageenan disaccharide: adding kappa-carrageenase Cgk-GDSX478 into a kappa-carrageenan solution, and performing enzymolysis to obtain the kappa-carrageenan disaccharide, wherein enzymolysis reaction conditions are: a temperature is 4-70° C., a pH is 3.0-11.0, and a time is 30-1440 minutes. The kappa-carrageenan disaccharide obtained by performing degradation on kappa-carrageenan by the kappa-carrageenase of the present disclosure has high singleness, which is of great significance for industrial production of the kappa-carrageenan disaccharides.

### CROSS-REFERENCE TO RELATED APPLICATION

This application claims the benefit of priority from Chinese Patent Application No. 202411788452.0 entitled “KAPPA-CARRAGEENASE CGK-GDSX478 FOR DIRECTIONALLY PREPARING KAPPA-CARRAGEENAN DISACCHARIDE” and filed on Dec. 6, 2024, the content of which is hereby incorporated by reference in its entire by reference.

### BACKGROUND

### Technical Field

The present disclosure generally relates to the field of biological enzyme technologies, and especially relates to a kappa-carrageenase Cgk-GDSX478 for directionally preparing a kappa-carrageenan disaccharide.

### STATEMENT REGARDING SEQUENCE LISTING

The substitute sequence listing is submitted as an XML file filed, with a file name of “CGK-GDSX478.XML”, a creation date of Sep. 10, 2024, and a size of 5,532 bytes. The substitute sequence Listing filed is a part of the specification and is incorporated in its entirety by reference herein.

### DESCRIPTION OF RELATED ART

As a sulfated polysaccharide, carrageenan has immunomodulatory, anti-tumor, antiviral and other effects. Carrageenan oligosaccharides are degradation products of carrageenan, which have advantages of lower molecular weight and higher solubility compared with carrageenan. At the same time, because their active groups are fully exposed, their activity is significantly improved compared with that before degradation, thereby showing good application prospects in the fields of biomedicine, food and agriculture. Due to diverse structural types of carrageenan, there are many connection methods and branching forms. The carrageenan oligosaccharides obtained through certain biotechnological treatments (such as physical degradation, oxidative degradation, acid degradation, enzyme degradation and molecular modification) have more complex structures of oligosaccharides, and the structures and activities of oligosaccharides obtained by different preparation methods are different.

The conventional technology mostly uses chemical hydrolysis to prepare carrageenan oligosaccharides. However, because the chemical hydrolysis reaction process is often too violent, it will cause destruction of a product structure during the hydrolysis process, which greatly limits an application thereof. A method of using carrageenase for specific degradation is considered to be a preparation method with broad prospects due to mild reaction conditions and good product specificity thereof. At present, the products for preparing carrageenan oligosaccharides by performing enzymatic hydrolysis on carrageenan are mostly disaccharides, tetrasaccharides and hexasaccharides, and even contain a small amount of octasaccharides. Therefore, the development of enzymatic hydrolysis technologies for preparing single products from carrageenan has an important research value and broad development prospects, and is also an important direction for the high-value development of the carrageenan industry.

The present disclosure uses the genome of Photobacterium rosenbergii GDSX-4 disclosed in Chinese Patent publication No. CN117247877A, titled “a strain of Photobacterium rosenbergii and an application in preparation of carrageenan oligosaccharides thereof” and published on Dec. 19, 2023, as a template to perform PCR amplification on an enzyme-producing gene thereof.

### SUMMARY

In view of the above related art, the present disclosure provides a kappa-carrageenase Cgk-GDSX478 that can degrade kappa-carrageenan and produce kappa-carrageenan disaccharides, wherein the enzyme can directionally prepare kappa-carrageenan disaccharides, which is of great significance for industrial production of the kappa-carrageenan disaccharides.

In order to solve the above objectives of the present disclosure, on one hand, a gene of an encoding kappa-carrageenase according to an embodiment of the present disclosure is provided, wherein the gene is Cgk-GDSX478, and a nucleotide sequence of the gene is shown in SEQ ID NO: 1.

Wherein the gene Cgk-GDSX478 is derived from the Photobacterium rosenbergii GDSX-4.

Furthermore, a recombinant expression vector according to an embodiment of the present disclosure is provided. The present disclosure is provided to respectively perform double enzyme digestion on Cgk-GDSX478 and an Escherichia coli expression vector pET-28a (+) by using restriction endonuclease (NdeI-xhoI), and then connects recovered fragments to the Escherichia coli expression vector with the same site of enzymatic cleavage to obtain the recombinant expression vector.

On the other hand, a recombinant engineered bacterium according to an embodiment of the present disclosure includes the above gene of encoding kappa-carrageenase or the above recombinant expression vector, wherein a host of the recombinant engineered bacterium is Escherichia coli, and wherein the Escherichia coli is BL21 (DE3).

Furthermore, in the present disclosure, BL21 (DE3) competent cells are transformed from recombinant expression vectors, coated on an LB agar plate with kanamycin resistance, and incubated overnight at a temperature of 37° C. Monoclonal antibodies are selected for performing positive validation and identification sequencing, and the monoclonal antibodies that are sequenced correctly are re-inoculated on onto the LB agar plates with kanamycin resistance to obtain the recombinant engineered bacterium.

On the other hand, the present disclosure provides a kappa-carrageenase that is produced by the above gene of encoding kappa-carrageenase, or by the above recombinant engineered bacterium, wherein the kappa-carrageenase is Cgk-GDSX478, and the amino acid sequence is shown in SEQ ID NO:2.

Furthermore, in the present disclosure, after the recombinant engineered bacterium is activated, the recombinant engineered bacterium is inoculated with 100 mL of LB sterile liquid medium that contains 50 μg/ml kanamycin at a volume ratio of 1% to expand the culture at the temperature of 37° C. and at a speed of 220 rpm, when the bacterial solution OD₆₀₀ is 0.5, 0.1 mM IPTG is added, and induced at a temperature of 25° C. for 12 hours. After the fermentation is completed, the culture solution is collected and centrifuged at a speed of 8000 rpm/min for 10 minutes to collect the thallus, the supernatant is taken through a filter membrane of 0.45 μm, and the crude enzyme solution is collected for performing protein purification by Ni-NTA affinity chromatography. The protein amino acid sequence that is purified is shown in SEQ ID NO: 2, and the relative molecular weight is about 35 kDa as detected by SDS-PAGE.

The present disclosure provides an application of the kappa-carrageenase for directionally preparing kappa-carrageenan disaccharides. An enzymatic hydrolysis temperature of the kappa-carrageenase is 4-60° C., an enzymatic hydrolysis pH is 3.0-11.0, and an enzymatic hydrolysis time is 30-1440 minutes. Preferably, the enzymatic hydrolysis temperature of the kappa-carrageenase is 40° C., the enzymatic hydrolysis pH is 8.0, and the enzymatic hydrolysis time is 420 minutes.

Furthermore, the present disclosure detects the enzyme activity of the kappa-carrageenase at the temperature of 4-60° C. and finds that the kappa-carrageenase Cgk-GDSX478 has good relative activity at the temperature of 4-60° C., and an optimal reaction temperature is 40° C. The present disclosure uses a buffer solution with a pH of 3.0-11.0 to dissolve carrageenan, and then adds the kappa-carrageenase Cgk-GDSX478 to detect the enzyme activity. It is found that the kappa-carrageenase Cgk-GDSX478 is relatively stable in the buffer solution system with the pH of 3.0-11.0, and can perform enzymatic hydrolysis, and the optimal reaction pH is 8.0.

Furthermore, the present disclosure uses the above-mentioned kappa-carrageenase Cgk-GDSX478 for performing enzymatic hydrolysis on the kappa-carrageenan to prepare kappa-carrageenan oligosaccharides. Through thin-layer chromatography and liquid phase mass spectrometry analysis, it is found that the enzymatic hydrolysis product after the kappa-carrageenan is performed enzymatic hydrolysis by the kappa-carrageenase Cgk-GDSX478 is the kappa-carrageenan disaccharide.

Compared with the related art, the technical solution provided by the present disclosure has at least the following beneficial effects or advantages:

The kappa-carrageenase Cgk-GDSX478 provided by the present disclosure has been experimentally researched to have good relative activity at the temperature of 4-60° C., and its optimal reaction temperature is 40° C. The kappa-carrageenase Cgk-GDSX478 is relatively stable in the buffer solution system with the pH of 3.0-11.0, and can perform enzymatic hydrolysis, and the optimal reaction pH is 8.0. Through thin-layer chromatography and liquid phase mass spectrometry analysis, it is found that the enzymatic hydrolysis product after the kappa-carrageenan is hydrolyzed by the kappa-carrageenase Cgk-GDSX478 is the kappa-carrageenan disaccharide. The above results indicate that the kappa-carrageenanase Cgk-GDSX478 has high degradation activity for the kappa-carrageenan, and the product has a high degree of singularity. The present disclosure is of great significance for the industrial production of the kappa-carrageenan disaccharide.

### BRIEF DESCRIPTION OF THE DRAWINGS

FIG. 1 is an agarose gel electrophoresis diagram of a gene Cgk-GDSX478 of the present disclosure.

FIG. 2 is an SDS-PAGE image of a kappa-carrageenase Cgk-GDSX478.

FIG. 3 is a diagram that shows an effect of different temperatures on the enzymatic hydrolysis reaction of the kappa-carrageenase Cgk-GDSX478.

FIG. 4 is a diagram that shows an effect of different temperatures on the incubation of the kappa-carrageenase Cgk-GDSX478.

FIG. 5 is a diagram that shows an effect of different pHs on the enzymatic hydrolysis reaction of the kappa-carrageenase Cgk-GDSX478.

FIG. 6 is a diagram that shows an effect of different pHs on the incubation of the kappa-carrageenase Cgk-GDSX478.

FIG. 7 is a diagram that shows an effect of different metal ions on the enzymatic hydrolysis reaction of the kappa-carrageenase Cgk-GDSX478.

FIG. 8 is a diagram that shows results of enzymatic hydrolysis products of the kappa-carrageenase Cgk-GDSX478 at different times by using a thin-layer chromatography analysis method, wherein Gal is galactose; DP2 is a standard product of the kappa-carrageenan disaccharide, DP4 is a standard product of the kappa-carrageenan tetrasaccharide, DP6 is a standard product of the kappa-carrageenan hexasaccharide, and 1 to 15 represent enzymatic hydrolysis reaction times of 10 minutes, 0.5 hour, 1 hour, 2 hours, 3 hours, 4 hours, 5 hours, 6 hours, 8 hours, 12 hours, 24 hours, 30 hours, 36 hours, 48 hours, and 72 hours, respectively.

FIG. 9 is a structural diagram of the enzymatic hydrolysis product of the kappa-carrageenase Cgk-GDSX478 detected by an ESI-MS, wherein DP2 is a standard product of the kappa-carrageenan disaccharide.

FIG. 10 is a mass spectrum of DP2, wherein DP2 is a monomer of the kappa-carrageenan disaccharide.

### DETAILED DESCRIPTION

The technical solution of the present disclosure is now described in conjunction with embodiments, however, the present disclosure is not limited to the following embodiments. The experimental methods and detection methods described in the following embodiments, unless otherwise specified, are conventional methods; reagents and materials described below are all commercially available unless otherwise specified.

### A First Embodiment

The first embodiment of the present disclosure provides a method for preparing the recombinant enzyme Cgk-GDSX478.

### 1. Acquisition of Gene Cgk-GDSX478

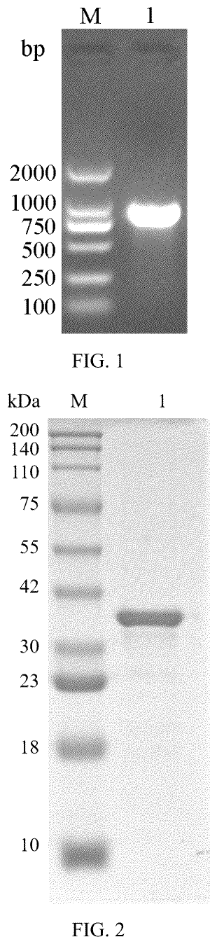

In an experiment, a genome of the Photobacterium rosenbergii GDSX-4 that is disclosed in Chinese patent publication No. CN 117247877A is taken as a template to perform PCR amplification on an enzyme-producing gene thereof. An upstream primer is designed to take an NdeI as the site 5′-GGAATTCCATATGACTTTAAACTTGTCTAATAA-3′, and a downstream primer is designed to take an xhoI as the site 5′-CCGCTCGAGCTCTCTGCTCCATACACGAACGTAC-3′. The PCR amplification system is 50 μL (2×pfu PCR mix 25 μL, 2 μL of each primer, 2 μL of the template, and 19 μL of ddH₂O), and amplification conditions are: a temperature of 94° C. for 5 minutes, 30 cycles (94° C. for 30 seconds, 55° C. for 30 seconds, 72° C. for 40 seconds) to be held on at a temperature of 10° C., to obtain the PCR product. The PCR product is detected by agarose gel electrophoresis, and the results are shown in FIG. 1. As shown in FIG. 1, the enzyme-producing gene Cgk-GDSX478 is amplified by PCR, with a size of 912 bp.

A preservation information of Photobacterium rosenbergii in the first embodiment is as follows:
a preservation date: Oct. 13, 2023;
a preservation unit: Guangdong Microbiological Culture Collection Center;
a preservation address: 5th Floor, Experimental Building, No. 100, Xianlie Middle Road, Yuexiu District, Guangzhou City, Guangdong Province; and
a preservation number: GDMCC No. 63878.

### 2. Acquisition of Recombinant Bacterium Cgk-GDSX478

The PCR products and the Escherichia coli expression vector pET-28a (+) (which are purchased from Hangzhou Baosai Biotechnology Co., Ltd.) are perform double enzyme digestion by using restriction endonuclease (NdeI xhoI), and the recovered fragments are ligated to the expression vector with the same site of enzymatic cleavage and transformed into BL21 (DE3) competent cells (purchased from Hangzhou Baosai Biotechnology Co., Ltd.), coated on the LB agar plates (kanamycin resistance), incubated overnight at a temperature of 37° C., and then Monoclonal antibodies are selected for performing positive validation and identification sequencing, and the monoclonal antibodies that are sequenced correctly are re-inoculated on onto the LB agar plates with kanamycin resistance to obtain the recombinant engineered bacterium Cgk-GDSX478.

### 3. Acquisition of Cgk-GDSX478 Enzyme

The recombinant bacterium Cgk-GDSX478 is activated in 5 mL of LB sterile liquid medium (containing 50 μg/mL kanamycin) and inoculated with 100 mL of LB sterile liquid medium (containing 50 μg/mL kanamycin) at a volume ratio of 1% to expand the culture at the temperature of 37° C. and at a speed of 220 rpm, when the bacterial solution OD₆₀₀ is 0.5, 0.1 mM IPTG is added, and induced at a temperature of 25° C. for 12 hours.

After the fermentation is completed, the culture solution is collected and centrifuged at a speed of 8000 rpm/min for 10 minutes, the supernatant is taken through a filter membrane of 0.45 μm, and the Cgk-GDSX478 crude enzyme solution is collected for performing protein purification by Ni-NTA affinity chromatography. The purified protein is detected by SDS-PAGE, and the results are shown in FIG. 2, which is a single band of electrophoretic pure protein, thereby indicating that Cgk-GDSX478 (kappa-carrageenase) is obtained, and a relative molecular weight thereof is about 35 kDa.

### A Second Embodiment

The second embodiment of the present disclosure provides the optimal enzymatic hydrolysis reaction conditions of the kappa-carrageenase Cgk-GDSX478 and the optimal incubation conditions of the Cgk-GDSX478 enzyme.

The method for determining the activity of the kappa-carrageenase Cgk-GDSX478 in the second embodiment is as follows: a substrate of 490 μL (0.2% kappa-carrageenan) and 10 μL of kappa-carrageenase Cgk-GDSX478 are reacted at a temperature of 40° C. for 30 minutes. After the reaction is complete, 500 μL of DNS reagent is added and mixed well, to boil the mixture for 10 minutes before color development.

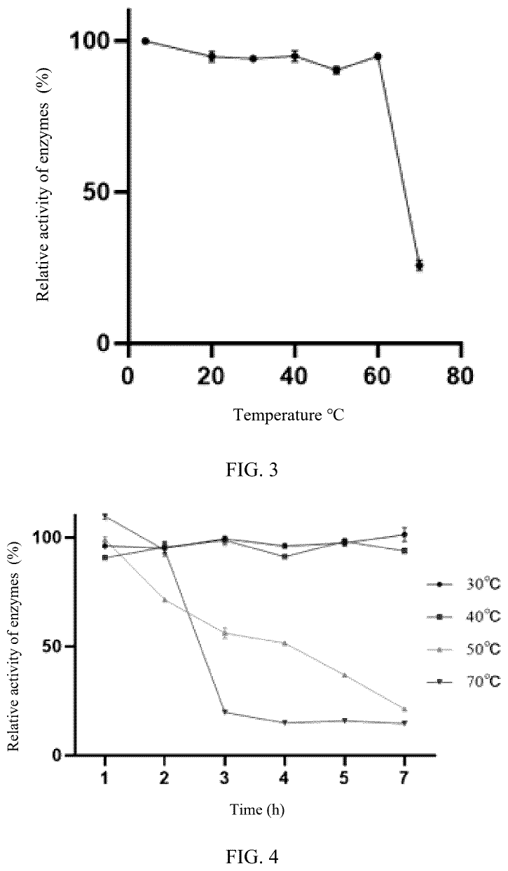

An enzymatic hydrolysis reaction temperature of the kappa-carrageenase Cgk-GDSX478 is: the kappa-carrageenase Cgk-GDSX478 and the kappa-carrageenan are subjected to perform enzymatic hydrolysis reaction at temperatures of 4° C., 20° C., 30° C., 40° C., 50° C., 60° C. and 70° C. The results are shown in FIG. 3. The enzymatic activity of the kappa-carrageenase Cgk-GDSX478 can be well at a temperature of 4-60° C., which achieves the degradation of the kappa-carrageenan.

An optimal temperature for performing the enzymatic hydrolysis reaction on the kappa-carrageenase Cgk-GDSX478 is: the kappa-carrageenase Cgk-GDSX478 is incubated at temperatures of 30° C., 40° C., 50° C. and 70° C. for 7 hours, to respectively measure the temperature stability of the kappa-carrageenase Cgk-GDSX478. The results are shown in FIG. 4. After incubation at temperatures of 30° C. and 40° C. for 7 hours, the activity of the kappa-carrageenase Cgk-GDSX478 can still retain more than 95%.

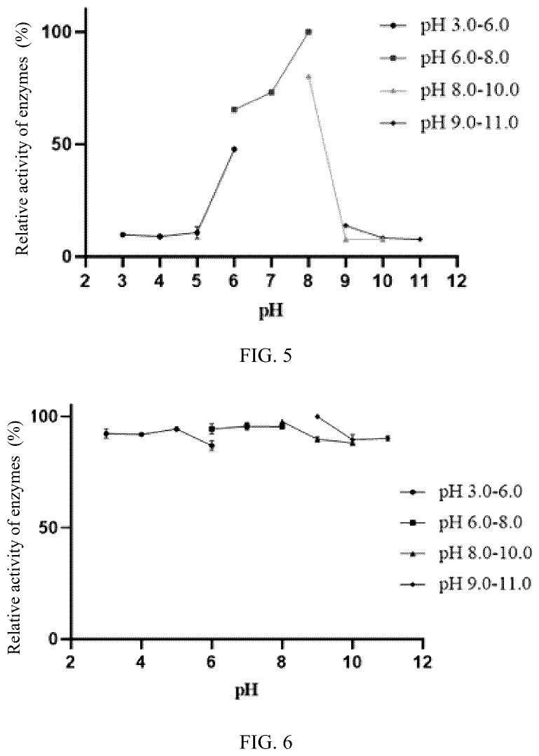

A pH for performing enzymatic hydrolysis reaction on the kappa-carrageenase Cgk-GDSX478 is as follows: at the temperature of 40° C., a buffer solution with a pH of 3.0 to 11.0 (the solution with pHs of 3.0, 4.0, 5.0 and 6.0 are citric acid buffer solution, the solution with pHs of 6.0, 7.0 and 8.0 are phosphate buffer solution, the solution with pHs of pH 8.0, 9.0 and 10.0 are Tris-HCl buffer solution, and the solution with pHs of 9.0, 10.0 and 11.0 are sodium carbonate buffer solution) is selected to dissolve kappa-carrageenan to test the optimal pH for performing enzymatic hydrolysis reaction on the kappa-carrageenase Cgk-GDSX478. The results are shown in FIG. 5, and the optimal pH for performing enzymatic hydrolysis reaction on the kapp-carrageenase Cgk-GDSX478 is 8.0.

The enzyme solution of the kappa-carrageenase Cgk-GDSX478 is mixed with the above-mentioned buffer solutions with different pHs and let it stand for 24 hours to determine the stability of the kappa-carrageenase Cgk-GDSX478 enzyme solution in buffer solutions with different pHs. The results are shown in FIG. 6. The kappa-carrageenase Cgk-GDSX478 enzyme solution is relatively stable in the buffer solution system with a pH of 3.0 to 11.0, and can play an enzymatic hydrolysis role thereof.

### A Third Embodiment

The third embodiment provides the effects of different metal ions on the enzymatic activity of the kappa-carrageenase Cgk-GDSX478.

An experimental group: Preparing metal ion reagents of Ni²⁺, Cu²⁺, Fe²⁺, Mn²⁺, Mg²⁺, Zn²⁺, Ca²⁺, K⁺, Ba²⁺ and Na⁺ with a final concentration of 5 mmol/L, to add a substrate of 490 μL (0.2% kappa-carrageenan) and 10 μL of kappa-carrageenase Cgk-GDSX478 for being reacted at a temperature of 40° C. for 30 minutes. After the reaction is complete, 500 μL of DNS reagent is added and mixed well, to boil the mixture for 10 minutes before color development.

A control group: 490 μL of substrate (0.2% kappa-carrageenan) and 10 μL of kappa-carrageenase Cgk-GDSX478 are reacted at the temperature of 40° C. for 30 minutes without adding metal ions as a control.

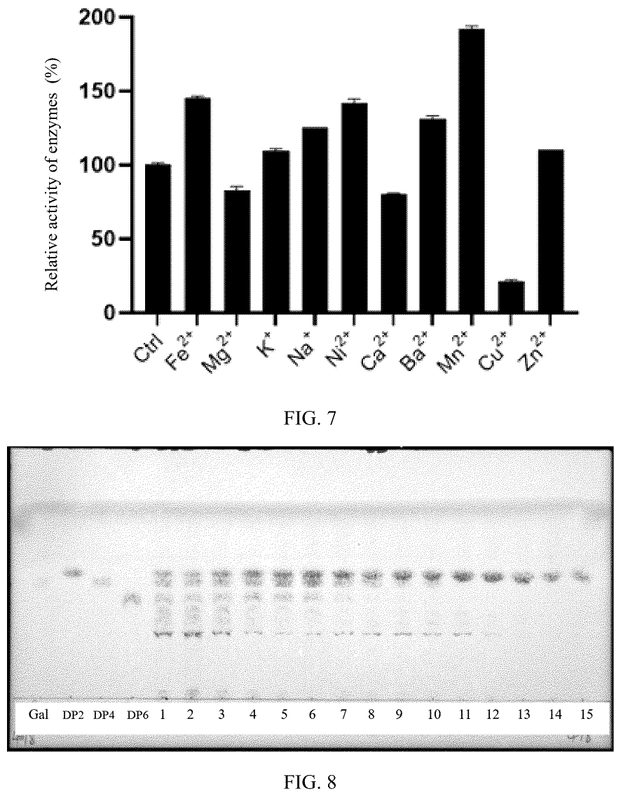

The results are shown in FIG. 7, and the metal icons Fe²⁺, Na⁺, Ni²⁺, Ba²⁺ and Mn²⁺ can all promote the activity of the kappa-carrageenase Cgk-GDSX478.

### A Fourth Embodiment

The fourth embodiment provides a method for preparing kappa-carrageenan oligosaccharides by using the kappa-carrageenanase Cgk-GDSX478 for performing enzymatic hydrolysis on the kappa-carrageenan.

### (1) Preparation of a Kappa-Carrageenan Oligosaccharide

A 0.5% of kappa-carrageenan substrate solution is prepared by using a phosphate buffer solution (a pH of 8.0), and 10% of kappa-carrageenanase Cgk-GDSX478 enzyme solution is added according to a total reaction volume thereof, to react at the temperature of 40° C. respectively for 10 minutes, 0.5 hour, 1 hour, 2 hours, 3 hours, 4 hours, 5 hours, 6 hours, 8 hours, 12 hours, 24 hours, 30 hours, 36 hours, 48 hours and 72 hours. After the reaction is completed, the above mixture is heated and boiled for 10 minutes, and then the heating is terminated and cooled to a room temperature to obtain a kappa-carrageenan oligosaccharide solution.

### (2) Analysis of the Kappa-Carrageenan Oligosaccharide by a Thin-Layer Chromatography Method

A silica gel thin-layer plate is taken and activated in a drying oven with a temperature of 100° C. for 1 hour, 3 μL of the above kappa-carrageenan oligosaccharide solution is taken and applied onto the silica gel thin-layer plate. Using galactose, standard disaccharides, tetrasaccharides, and hexasaccharides as controls and developing into a developing agent of N-butanol:ethanol:water (3:2:2). After completing development, blowing dry the silica gel thin-layer plate. After soaking in a chromogenic reagent (2 g of diphenylamine, 2 mL of aniline, 10 mL of 85% phosphoric acid, 1 mL of concentrated hydrochloric acid, 100 mL of acetone), blowing dry the silica gel thin-layer plate, and finally heating the silica gel thin-layer plate at a temperature of 110° C. for 10 minutes for color development.

As shown in FIG. 8, the results indicate that the product of the kappa-carrageenan is performed enzymatic hydrolysis by the kappa-carrageenase Cgk-GDSX478 is mainly the carrageenan disaccharide.

### (3) Analysis of a Degradation Product Structure by a Liquid Chromatography-Mass Spectrometry (Q-TOF-MS)

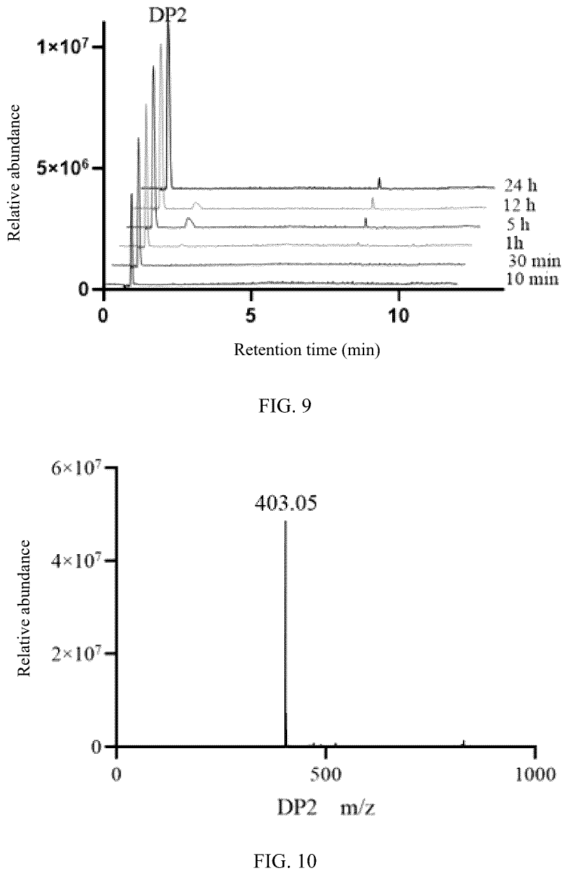

The kappa-carrageenan oligosaccharide solution is diluted to 50 μg/mL, and filtered through a 0.22 μm of microporous filter membrane, and injected into the TOF-MS system for analysis, wherein chromatographic conditions are: an injection volume is 5 μL, a mobile phase is acetonitrile: 1 mM formic acid water=1:1, a flow rate is 0.2 mL/min, and a column temperature is 35° C. The mass spectrum adopts an ESI point spray ion source, and a negative ion ionization mode; a mass scanning range is: m/z 50-2000 Da. The ESI-MS detection results are shown in FIG. 9, which indicates that the product of the kappa-carrageenan is performed enzymatic hydrolysis by the kappa-carrageenase Cgk-GDSX478 is only the carrageenan disaccharide.

The embodiments described above are part of embodiments of the present disclosure, but not all embodiments of the present disclosure. The detailed description of the embodiments of the present disclosure is not intended to limit the scope of the present disclosure claimed for protection, but only represents selected embodiments of the present disclosure. Any variation or replacement made by one of ordinary skill in the related art without departing from the spirit of the present disclosure shall fall within the protection scope of the present disclosure.

What is claimed is:

1. A gene of an encoding kappa-carrageenase comprising: wherein the gene is Cgk-GDSX478, and a nucleotide sequence of the gene is shown in SEQ ID NO: 1; and wherein the gene Cgk-GDSX478 is derived from the Photobacterium rosenbergii GDSX-4.

2. A recombinant expression vector comprising the gene of encoding kappa-carrageenase as claimed in claim 1, wherein the gene of encoding kappa-carrageenase is linked to an expression vector to obtain the recombinant expression vector.

3. A recombinant engineered bacterium comprising the gene of encoding kappa-carrageenase as claimed in claim 1 or the recombinant expression vector as claimed in claim 2.

4. The recombinant engineered bacterium as claimed in claim 3, wherein a host of the engineered bacterium is Escherichia coli, and wherein the Escherichia coli is BL21.

5. A kappa-carrageenase is produced by the gene of encoding kappa-carrageenase as claimed in claim 1, or by the recombinant engineered bacterium as claimed in claim 3, wherein the kappa-carrageenase is Cgk-GDSX478, and an amino acid sequence is shown in SEQ ID NO:2.

6. An application of the kappa-carrageenase as claimed in claim 6 for preparing kappa-carrageenan disaccharides is provided.

7. The application as claimed in claim 6, wherein an enzymatic hydrolysis temperature of the kappa-carrageenase is 4-60° C., an enzymatic hydrolysis pH is 3.0-11.0, and an enzymatic hydrolysis time is 30-1440 minutes.

8. The application as claimed in claim 7, wherein an enzymatic hydrolysis temperature of the kappa-carrageenase is 40° C., an enzymatic hydrolysis pH is 8.0, and an enzymatic hydrolysis time is 420 minutes.
